(19) United States
(12) Patent Application Publication
(10) Pub. No.: US 2022/0022800 A1
(43) Pub. Date: Jan. 27, 2022

(54) MONITORING BASED ON CONTINUOUS INTRACRANIAL EEG ACTIVITY

(71) Applicant: Cerebral Therapeutics, Inc., Aurora, CO (US)

(72) Inventors: Daniel J. Abrams, Denver, CO (US); Aaron Reite, Aurora, CO (US)

(21) Appl. No.: 17/380,694

(22) Filed: Jul. 20, 2021

Related U.S. Application Data
(60) Provisional application No. 63/054,522, filed on Jul. 21, 2020.

(51) Int. Cl. A61B 5/37; A61B 5/293; A61B 5/372; A61B 5/00

(52) U.S. Cl. CPC A61B 5/37; A61B 5/293; A61B 5/372; A61M 27/006; A61B 5/6852; A61B 5/7275; A61B 5/746; A61B 5/4839

(57) ABSTRACT

A method receives EEG data from at least one electrode implanted in the brain of the subject. The method determines a current or predicted brain state from the EEG data using an artificial intelligence (AI) model

### RELATED APPLICATION

This application claims the benefit of U.S. Provisional Patent Application No. 63/054,522, filed on Jul. 21, 2020, which application is hereby incorporated herein by reference in its entirety to the extent that it does not conflict with the disclosure presented herein.

### FIELD

The present technology is generally related to methods, devices, and systems to understand, monitor, and/or treat a disease of a subject based on electroencephalogram (EEG) data.

### INTRODUCTION

Brain activity is difficult to study because of the inherent complexity of the brain, which includes billions of neurons and trillions of synaptic connections. Existing methods, such as magnetic resonance imaging (MRI), functional MRI, electroencephalogram (EEG), and magnetoencephalogram (MEG) recordings, are unable to measure high quality temporally relevant brain data over long periods of time and in real time.

Current EEG techniques involve recording brain activity from electrodes placed on the scalp. Such EEG recordings may be useful for periodic monitoring of the patient's brain activity but are not suitable for long-term, continuous monitoring for patients that are not confined to a health care facility. In addition, the signal provided by such EEG recordings tends to be noisy and of lower quality due to signal attenuation through the skull and scalp.

### SUMMARY

The present disclosure relates to, among other things, methods, devices, and systems that identify or utilize biomarkers of electrical activity associated with a disease of the brain or associated with another brain state, based on long-term high-quality real time intracranial EEG (iEEG) data. The iEEG data may be continuous iEEG (ciEEG) data. The iEEG data may be obtained from electrodes placed within the brain of a subject. By placing the electrodes within or in proximity to brain tissue, such as white matter or grey matter, less “noisy” signals may be obtained than with scalp-based EEG recordings. Such higher quality signals may facilitate Artificial Intelligence (AI) processing of data recorded by the electrodes to identify electrical activity biomarkers associated with the brain disease, to determine a brain state, or to predict a future brain state.

Artificial intelligence (AI) techniques may be used to generate an AI model, such as a deep neural network (DNN), associated with the subject or the disease. By using high quality iEEG data the DNN may more readily identify biomarkers associated with the brain disease, determine the brain state, or predict the future brain state. The AI model may be used for any suitable purposes, such as for monitoring a disease or therapy progression.

Once the AI model is established using high quality iEEG data, the AI model may be applied to, and potentially refined with, lower quality data, such as that obtained by scalp-based EEG data. Once electrical biomarkers and electrical activity indicative of a brain state are established with the high-quality iEEG data, such biomarkers and electrical activity, or derivatives thereof, may be identified from more noisy data, even if the biomarkers and electrical activity may not have been originally identifiable from the noisy data itself. Accordingly, the DNNs or AI models established according to the methods described herein may be non-invasively applied to, or refined by non-invasively collected scalp-based EEG data from, subjects suffering from, or at risk of, a brain disease for which the DNN or AI model has been established.

According to an aspect of the present disclosure, a method comprises receiving electroencephalogram (EEG) data from at least one electrode implanted in a brain of a subject; and establishing an artificial intelligence (AI) model to identify an electrical signal biomarker associated with a brain state from the EEG data. The electrode may be implanted in white matter or grey matter of the brain. Preferably, the electrode is implanted in white matter of the brain.

The established AI model may be applied to identify an electrical signal biomarker associated with a brain state based on nascent collected data, such as EEG or MEG data, which may be collected in real time or near real time.

The established AI model may predict a future brain state based on the nascent collected data. A subject may be notified of the predicted future brain state.

The established AI model may be used to adjust administration of a therapy to the brain of the subject.

According to an aspect of the present disclosure, a method comprises receiving electroencephalogram (EEG) data from at least one electrode implanted in a brain of a subject; and determining a brain state or predicting a future brain state of the subject based on the EEG data using an artificial intelligence (AI) model. The brain state may be associated with a disease of the brain of the subject. The electrode may be implanted in white matter or grey matter of the brain. Preferably, the electrode is implanted in white matter of the brain.

The AI model may be applied to identify an electrical signal biomarker associated with a brain state based on nascent collected data, such as EEG or MEG data, which may be collected in real time or near real time.

The AI model may predict a future brain state based on the nascent collected data. A subject may be notified of the predicted future brain state.

The AI model may be used to adjust administration of a therapy to the brain of the subject.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1 is a flow diagram illustrating an embodiment of a method for establishing and refining an AI model.

FIG. 2 is a flow diagram illustrating an embodiment of a transfer learning process.

FIG. 3 is a graphical representation space and unsupervised cluster arrangement provided to an unsupervised deep neural network trained on raw, unlabeled EEG data.

FIG. 4 is a graphical representation of a 2-D visualization of an unsupervised representation space, colored by sleep stages.

FIG. 5 is a flow diagram illustrating an embodiment of a method for employing an AI model.

FIG. 6 is a schematic diagram illustrating an embodiment of a system for collecting data for use in establishing or refining an AI model.

FIG. 7 is plot illustrating relationships between biomarkers, brain states, and different types of treatment.

FIG. 8 is a schematic block diagram illustrating an embodiment of a system that may collect data that may be used by an AI model to adjust therapy delivery.

While the invention is susceptible to various modifications and alternative forms, specific embodiments thereof are shown by way of example in the drawings and may herein be described in detail. The drawings may not be to scale. It should be understood, however, that the drawings and detailed description thereto are not intended to limit the invention to the particular form disclosed, but on the contrary, the intention is to cover all modifications, equivalents and alternatives falling within the spirit and scope of the present invention as defined by the appended claims.

Like numbers used in the figures refer to like components and steps. However, it will be understood that the use of a number to refer to a component in a given figure is not intended to limit the component in another figure labeled with the same number. In addition, the use of different numbers to refer to components in different figures is not intended to indicate that the different numbered components cannot be the same as or similar to other numbered components DETAILED DESCRIPTION

The present disclosure relates to, among other things, methods, devices, and systems that identify or utilize biomarkers of electrical activity associated with a disease of the brain or associated with another brain state. The identification of electrical biomarkers associated with a disease of the brain or another brain state may be based on long-term, continuous, high-quality, real-time intracranial EEG (ciEEG) data. The ciEEG data may be obtained from electrodes placed within the brain of a subject. The electrodes may be placed in white matter or grey matter of the brain of a subject. Preferably, the electrodes are placed in white matter.

White matter of the brain is composed mainly of long-range myelinated axons and serves as a preferred target for monitoring general electrical activity of the brain. The electrodes in the white matter may capture electrical activity associated with general brain state rather than merely capturing signals localized to small regions of the brain. The high-quality brain activity signals recorded by electrodes in white matter may facilitate processing (e.g., interpretation and analysis) of the iEEG data.

Grey matter of the brain is composed mainly of neuronal cell bodies. The electrodes in grey matter may primarily capture local field potentials, or electrical activity associated with local regions of brain. Recording activity within confined brain regions, or interactions between two or more grey matter regions may facilitate processing of the iEEG data.

Whether the electrodes are placed in white matter, grey matter, or both white matter and grey matter, the electrodes are preferably placed and configured to record electrical activity associated with general brain activity or are capable of recording electrical activity that may be processed to determine general brain activity, rather than merely recording local field potentials or electrical activity associated with a local region of the brain without the ability to determine more general brain activity.

Preferably, the iEEG data is collected from subjects that are suffering from a disease for which treatment indicates the need for implantation of a device, such as a lead or a catheter, in the subject's brain. The implantation of the electrodes in the brain may occur as a part of the procedure to implant the treatment device in the brain of the subject. Accordingly, the subjects need not undergo a separate second invasive procedure to implant the recording electrodes.

Examples of therapeutic devices that may be implanted in a brain of a subject include deep brain stimulation (DBS) leads and catheters. The catheters may be used to deliver therapeutic agent to the brain, such as the brain parenchyma or to a cerebrospinal fluid (CSF)-containing space of the brain, or to drain CSF from the brain. Examples of diseases that may be treated by DBS include dystonia, epilepsy, essential tremor, and Parkinson's disease. Examples of diseases that may be treated with a CSF drainage catheter include subarachnoid hemorrhage, intracerebral hemorrhage, subdural hemorrhage, extradural hemorrhage, obstructive hydrocephalus, nervous system cancer (such as secondary malignant neoplasm of the brain, spinal cord, or other parts of the nervous system; malignant neoplasm of the cerebellum nos; malignant lymphoma of an unspecified site, extranodal site, or solid organ site; and the like), cerebral artery occlusion, unspecified cerebral infarction, closed fracture of the base of the skull (which may be associated with one or more of subarachnoid hemorrhage, subdural hemorrhage, extradural hemorrhage, and loss of consciousness), infection and inflammatory reaction (such as due to nervous system device, implant or graft), mechanical complication of a nervous system device, implant or graft), traumatic brain injury, congenital hydrocephalus, aneurysms, and the like. The CSF drainage catheter may be an external ventriculostomy drainage catheter, a ventriculoperitoneal shunt, or the like. Diseases that may be treated by delivery of therapeutic agents to the brain include epilepsy, bipolar disorder, depressive disorder spectrum, anxiety disorder spectrum including post-traumatic stress disorder (PTSD), cognitive disorder spectrum, memory disorder spectrum, processing speed disorder spectrum Parkinson's disease, Alzheimer's disease, dementia, Amyotrophic Lateral Sclerosis, Huntington's disease, lysosomal storage diseases, post-traumatic stress disorder, anxiety, depression, brain tumors, autism, autism spectrum disorder, stroke, schizophrenia, brain infection, and the like.

Preferably, the iEEG data is collected from subjects that are suffering from a disease for which treatment indicates the need for implantation of a catheter, such as a CSF drainage catheter or a therapeutic agent delivery catheter, in the subject's brain. Preferably, the therapeutic agent delivery catheter is a catheter configured to deliver the therapeutic agent to a CSF-containing space of the brain, such as a cerebral ventricle. Currently used CSF drainage catheters and therapeutic agent delivery catheters do not have any means for recording electrical activity, and thus iEEG recording is not possible. While electrodes of DBS leads may be used to record iEEG data, the electrodes are typically configured to record local field potentials and not used to record or process data regarding more global brain activity, which is more indicative of a brain state. If a DBS lead is implanted in a subject, a separate recording electrode may implanted to record more global brain activity or the DBS lead may be modified to configure the electrodes such that they may be used to monitor more global brain activity.

In some embodiments, the data is recorded from a catheter, such as a CSF drainage catheter or therapeutic agent delivery catheter, that has been modified to include one or more recording electrodes or to a catheter to which a lead having recording electrodes is coupled. A single implantation step may result in implantation of such catheters and recording electrodes, whether associated with the catheter or a coupled lead, rather than two separate implantation steps, which may occur during the same surgical procedure. Examples of catheters modified to include recording electrodes or coupled to leads having recording electrodes are disclosed in U.S. Provisional Patent Application No. 63/053,864, filed on Jul. 20, 2020, and U.S. patent application Ser. No. 17/380,415, filed on Jul. 20, 2021, which patent applications are hereby incorporated herein by reference in their respective entireties to the extent that they do not conflict with the disclosure presented herein. Capturing recorded electrical signals in subjects receiving treatment via a catheter placed in the brain without additional substantial surgical complexity or invasiveness may provide meaningful advancement in the understanding of brain states in general and brain states of subjects suffering from brain diseases in particular.

While the iEEG data, preferably ciEEG data, may be collected from a subject suffering from a disease for which treatment indicates the need for implantation of a device in the subject's brain, the data may be used to establish AI models unrelated to the disease being treated. For example, iEEG data from a subject suffering from epilepsy may be useful in establishing AI models associated with anxiety, depression, or any other disease or other brain state experienced by the subject during non-epileptic periods.

Preferably, the iEEG data is used to establish AI models related to the disease being treated. The established AI models may be used to monitor the disease being treated, monitor therapy progression, enhance therapy, facilitate patient care, or for any other suitable purpose. For example, if the iEEG data is collected from a subject in which a therapeutic agent delivery catheter has been implanted, the AI model may be used to control or determine the rate of delivery of a therapeutic fluid. In some embodiments, treatment may include various phases that may be repeated in a cycle to manage treatment, such as observation and data collection, application of the AI model, treatment based on the AI model, and repeating as needed.

The established AI models may provide meaningful insight into a brain state of a subject. For example, the AI models may determine a current brain state or predict a future brain state based on the iEEG data. As used herein, the term “brain state” refers to a symptom (abnormal or normal) or psychological function that is psychologically known to the subjects and may have normal or abnormal manifestations. Brain states may impact one or more areas of the brain. In some cases, brain states may be referred to as episodes or events. Non-limiting examples of brains states include: normal, seizure, traumatic memory recall, suicidal thoughts, manic episode in bipolar disorder, anxiety in general anxiety disorder, sad feelings in major depressive disorder, etc. A brain state may be determined by an AI model, for example, based on iEEG activity indicative of the symptom or psychological function.

While the AI models described herein may be useful for identifying and employing electrical signal biomarkers and brain states associated diseases such as epilepsy and movement disorders such as Parkinson's disease, the AI models established from EEG data described herein may also provide meaningful insight into brain states of subjects suffering from diseases of the brain that are more in the psychiatric realm than in the neurology realm, and do not involve substantial electrical activity associated with movement. Such conditions and brain states are not very well understood but are most closely related to psychiatric disorders and leading psychiatric symptoms.

Recording brain EEG activity has not been utilized successfully in such psychiatry and psychology subjects even by using a separate system to periodically record brain activity from the scalp as has used for epilepsy and sleep. Accordingly, higher quality iEEG signals that are collected and analyzed over the long term may facilitate AI modelling because of the quality and length of recording. In addition, the methods, devices, and systems described herein, may provide for more meaningful AI models of epilepsy and sleep due to the length and quality of iEEG recordings, such as recordings from electrodes placed in white matter or grey matter. Various AI techniques may be paired with the vast amount of data recorded from the white or grey matter of the brain to identify electrical biomarkers associated with a brain disease or indicative of another brain state of a subject that would not typically be available from using only surface electrodes in an EEG.

The AI models described herein may employ any suitable AI technique. For example, one or more of unsupervised, semi-supervised, and transfer learning techniques may be employed to establish the model or to refine the model. Such techniques may be particularly effective when large amounts of ciEEG activity data from the white or grey matter of the brain are available. The AI model may be established on or refined by data obtained from the brain of a single subject or a population of subjects.

The AI model may comprise a DNN. A DNN is a sub-field of machine learning which leverages a composition of many nonlinear functions to map input data into a new desired output domain. The parameters of these nonlinear functions are not directly designed by humans, but instead learned from vast quantities of data. This allows the continual learning and improvement of a DNNs performance through the collection of more high-quality data. DNNs have found widespread success across numerous domains that often match or surpass human performance on specific tasks. Initially, deep learning strategies may be developed for iEEG analysis associated with waking EEG classification for emotions, motor activity, cognitive activity, seizure detection, a disease event, and sleep scoring including convolutional neural networks and recurrent neural networks. The initial strategies may utilize one or more of unsupervised, semi-supervised, and transfer learning approaches. The course of development of the DNN may include ongoing analysis iEEG data subject to ongoing analysis by modern machine learning techniques as well as classical methods such as support vector machine and decision tree methods.

After initial AI modelling has been sufficiently refined from long term high-quality iEEG recordings, the AI modelling (e.g., the learned aspects and algorithms) may be more meaningfully applied to lower quality external shorter-term (e.g., scalp) EEG recordings.

As used herein, the term “administer treatment” refers to providing information to the subject or physician (such as a notification) or to control a device or system to automatically provide treatment to the subject (such as drug infusion).

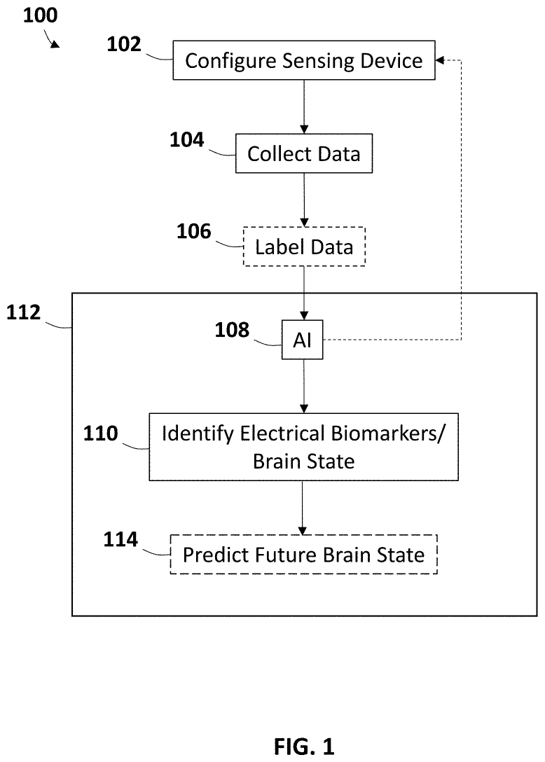

Referring now to FIG. 1, an example of a method 100 of using artificial intelligence techniques to establish or refine an AI model 112 is shown. In general, the method 100 includes collecting data indicative of activity in white or grey matter of a brain of a subject and identifying electrical biomarkers associated with a disease or another brain state based on that activity.

The method 100 may include configuring a sensing device in block 102. The sensing device may include any suitable device configured to record parameters related to the white or grey matter of the subject. In some embodiments, the sensing device may include one or more electrodes configured to record electrical activity, particularly electrical activity, of the white or grey matter.

The sensing device may be implantable or non-implantable. The sensing device may include a surface EEG device or a surface MEG device configured to record the respective data related to the subject. The sensing device may include an implantable portion and a non-implantable portion. Preferably, the sensing device is initially at least partially implantable such that recording electrodes are placed in contact with white matter or grey matter of the brain, preferably white matter.

In some embodiments, a sensing device comprises multiple recording electrodes. By looking at impact on multiple recording electrodes in the brain at the same time, rather than a single electrode, data may be provided for relevant non-local electrical activity. For example, multiple electrodes may be used to monitor activity related to any kind of brain state, which may be processed, analyzed, and parsed for a signal biomarker. The collected data from the multiple electrodes may be used to confirm a broader impact on the brain than would be detectable using a single electrode in a single location. Such use of multiple electrodes may enable determination of whether a consistent pattern between different electrodes, and thus a potential broader impact on the brain, is present in a subject. Data indicative of the broader impact may be helpful in indicating a broader brain state, which may reflect a measure associated with an underlying disease. For example, broader brain state AI models may be used to monitor psychiatric or psychological states, which may be related to an underlying psychological or psychiatric symptom. In one example, a broader brain state that may be detected is mania in bipolar disorder or a traumatic recall in PTSD.

In some embodiments, the broader brain state may be used to indicate whether treatment is improving the brain state. The broader brain state may also indicate whether other conditions in the brain are occurring, which may not be directly related to an administered treatment.

The method 100 may also include collecting data using the sensing device in block 104. Data collected by the sensing device may include information about activity of the white or grey matter. In particular, brain activity data indicative of activity in white or grey matter of the brain of the subject may be collected, recorded, or received. In some embodiments, long term ciEEG data may be collected using an implantable device.

The method 100 may also include an optional labeling of data in block 106. Labeled data may be used as inputs to certain AI models. As used herein, the term “labeling data” refers to data generated by tagging the collected data based on manual input or sensor input, for example, by a subject or a physician. In one example, collected data may include electrical activity data of the subject's brain over time and labeled data may include the collected data and tagged data indicative of a brain state, such as when experiencing a recalled post traumatic memory or when experiencing mania, a symptom of bipolar disorder, marked at a time stamp or during a period of time relative to the electrical activity data. The collected data may be labeled by marking. In some embodiments, a subject, physician, or the like may manually mark a brain state observed, such as a seizure state, for example, by pushing a button. In some embodiments, sensor data may be used to mark collected data. Non-limiting examples of other data that may be measured and used to mark collected data of the brain may include: a change in an accelerometer (e.g., subject activity or motion), heart rate, visual stimuli, skin conductance, audio or video recordings, or other physiological parameters.

Labeling can be done manually or by other wearables, such as a visual glasses, heart rate monitor, skin conductance monitor, respiration, temperature, or actimetry or accelerometer mobility and position monitor (e.g., configured to determine whether a subject falls, gets up from bed, goes for a walk, etc.). Such information may be used as inputs to the AI model. The AI model may use the information directly or may further label the collected data with specific activities. Non-limiting examples of labels for activities may include: arising from bed, going to the bathroom, starting a meal, or meeting a certain person. The labeling may be performed in real time, near real time, or retrospectively.

In some embodiments, labeling may be performed by time stamping. A subject may actuate a device to record the time whenever the subject has a feeling or thought or inclination for a behavior, such as post-traumatic stress disorder (PTSD)-related memory recall or anxiety associated with that memory, bipolar-related starting to feel manic, or suicide-related feelings of suicide start.

Collected data may be curated based on the labeled data. Curation may be done manually and turned into a series of rules that act as a filter for the collected data. In one embodiment, curation may be performed for PTSD when a memory is triggered. For example, a memory may be triggered for a war veteran subject by seeing restaurant decorations including scenes of the country where the subject served (e.g., visual stimuli) or having similar smells from the same country (e.g., olfactory stimuli). A video-recording headwear (e.g., recording glasses) may be used to record those visual or olfactory stimuli to provide marking and curation in a manual or automated manner. When the visual stimulus is detected, the AI model may begin to recognize a brain state without solely relying on brain activity data, such as the ciEEG data. Such a curation technique could be applied to other spontaneous events for other diseases, such as alcohol addiction.

Some AI models may not use, or need, labeled data.

The method 100 may further include establishing or refining an artificial intelligence (AI) model in block 112 by applying AI techniques in block 108. In general, an AI model, such as a DNN, may be trained to identify electrical biomarkers associated with a disease or another brain state 110 and used to determine correlations between one or more biomarkers in collected data and one or more brain states.

In some embodiments, the AI model may be used to identify one or more biomarkers 110 in the collected data. The biomarkers may be indicative of brain states in the collected data. Further, the AI model may be used to identify the onset of the brain states based on the collected data, for example, using the identified biomarkers 110 and may optionally predict future brain states 114 based on the identified biomarkers.

Any suitable AI technique or combination of techniques may be used to establish or refine the AI model. In some cases, the AI model may involve deep learning and a DNN. In some embodiments, a contrastive learning model or network may be used. The contrastive learning model may be used on unlabeled or partially labeled data in an unsupervised or semi-supervised manner. The contrastive learning model may include one or more parameters to facilitate learning. In some embodiments, the contrastive learning model may be configured to identify one or more biomarkers associated with a disease or other brain states based on time between different segments. In one example, a first duration may be calculated between a first data segment and a second data segment of the brain activity data. The first data segment and the second data segment may be determined to correspond to a first brain state. A third data segment of the brain activity data may be determined to correspond to a second brain state different than the first brain state in response to a second duration between the first data segment and the third data segment being greater than the first duration.

In some embodiments, the AI model is trained using supervised machine learning. In particular, supervised machine learning may be used when the collected data is labeled. Labeling data from the subject, a physician, or the like may be used to label a biomarker associated with a disease or other brain state relative to brain activity data and the AI model may be trained based on the labeling data and the brain activity data.

In other embodiments, the AI model is trained using unsupervised machine learning. For example, unsupervised machine learning may be used when the collected data is unlabeled. Relevant features in the collected data may be automatically learned and extracted using unsupervised learning, and clusters corresponding to brain states may emerge naturally using these techniques. In general, the unlabeled collected set of data may be much larger than a labeled set of data to appropriately train the AI model. In still other embodiments, the AI model may use semi-supervised learning. In one example, only some of the collected data may be labeled.

The AI model may be first trained using unsupervised or semi-supervised feature learning to identify biomarkers associated with a disease or other brain states from electrical signal data. The AI model may be second trained using different data to fine tune the AI model after unsupervised or semi-supervised feature learning to train the AI model. The different data may include, for example, labeled data versus unlabeled data. Labeled data may be used for supervised feature learning to fine tune the AI model.

Transfer learning may also be used to train the AI model. In some embodiments, the AI model may first be trained on a large set of unlabeled data, such as activity of the white or grey matter of one or more subjects. The AI model may second be trained on a smaller set of labeled data to fine tune the AI model, for example, for one particular subject. Transfer learning is represented by illustration as the line from block 108 to block 102, in which after the AI model is trained, the same sensing device or another sensing device may be configured to collect data to further train or refine the AI model.

Unsupervised feature learning may benefit from using very large volumes of diverse data to train the AI model. For example, brain activity data related to the white matter, grey matter, or both may be used to provide the diverse data. Transfer learning may benefit from using robust feature extractors trained on very large volumes of diverse data.

The AI model may be further trained using transfer learning based on data collected from different sensors. In one example, the AI model may first be trained based on data collected from one or more electrodes implanted in one or more subjects. The model may then be trained on, for example, 7-day EEG or MEG data from a subject. This approach may be useful for helping to treat subjects without implants using data from subjects with implants.

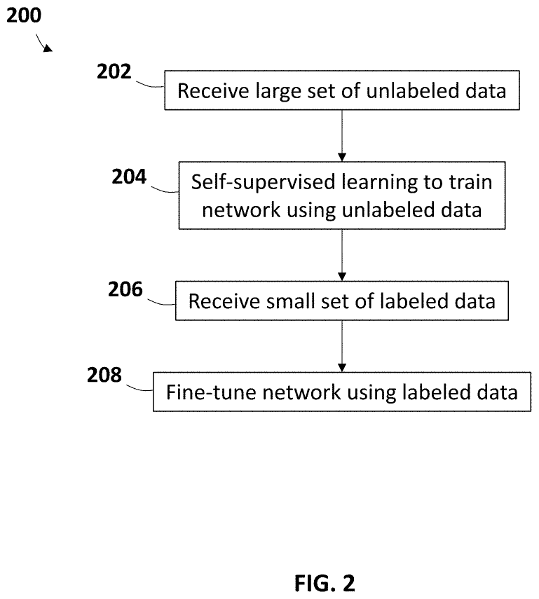

FIG. 2 shows one example of a method 200 of using transfer learning. The method 200 may include receiving a large set of unlabeled data in block 202. Self-supervised learning may be used to train an AI model or network using unlabeled data in block 204. A small set of labeled data may be received in block 206. The AI model or network may be fine-tuned using the labeled data for a specific treatment in block 208.

Data other than brain activity data indicative of activity in white or grey matter of the brain may be used to determine or predict a brain state. In some embodiments, other data used to supplement the EEG data may include one or more of the following types of data corresponding to the subject: activity, motion, heart rate, visual stimuli, audio or video recordings, and skin conductance.

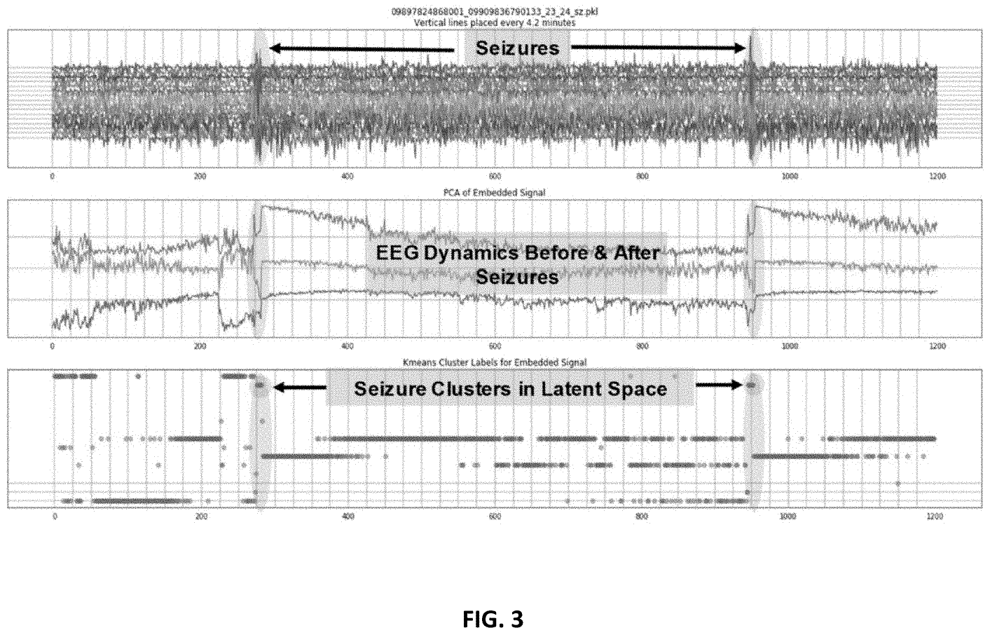

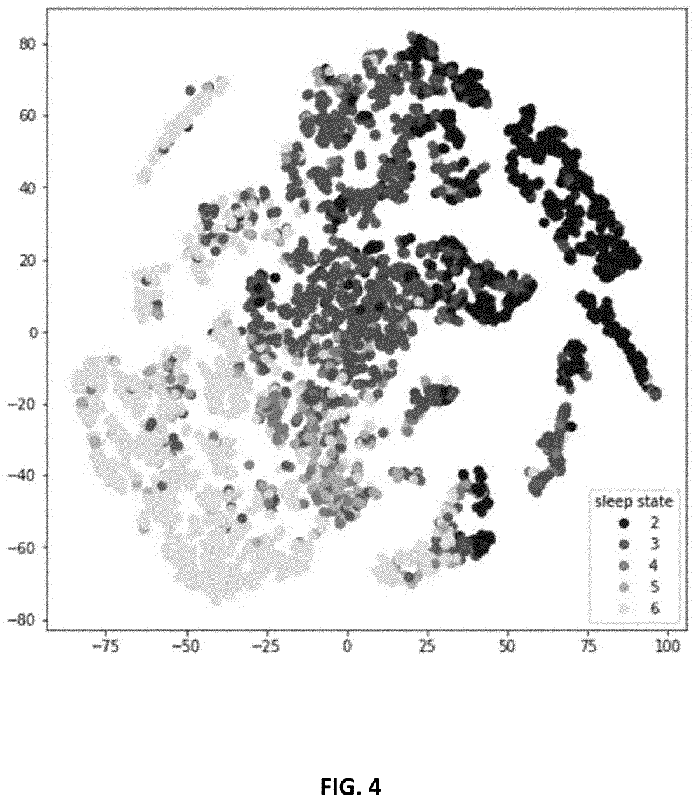

During training of the AI model, a visual representation may be generated to facilitate learning, particularly in lower dimensions than the full set of data collected. In some embodiments, a visual representation of multidimensional brain activity data in a lower-dimensional data structure may be generated. Labeling data to label a brain state relative to the visual representation of the lower-dimensional data structure may be received, for example, from a physician or other user facilitating training of the AI model. The AI model may be further trained based on the labeling data. In one example, raw data may be provided to a deep neural net, to a self-supervised pretext task, and to a low-dimensional representation space for evaluation. FIG. 3 shows one example of a representation space and unsupervised cluster arrangement provided to an unsupervised deep neural network trained on raw, unlabeled EEG data. FIG. 4 shows a 2-D visualization of an unsupervised representation space, colored by sleep stages.

One approach to AI model learning may utilize millions of free labels, which may be used to train a feature extractor with a pretext task. One channel in the collected data may be used to predict another. For example, one channel (e.g., from a first electrode) may be used to predict features in another channel (e.g., from a second electrode). Two events (e.g., two biomarkers) may also be used to predict the occurrence of an event (e.g., a biomarker or brain state) in between. Further, limited labeling data may be used to fine tune the AI model.

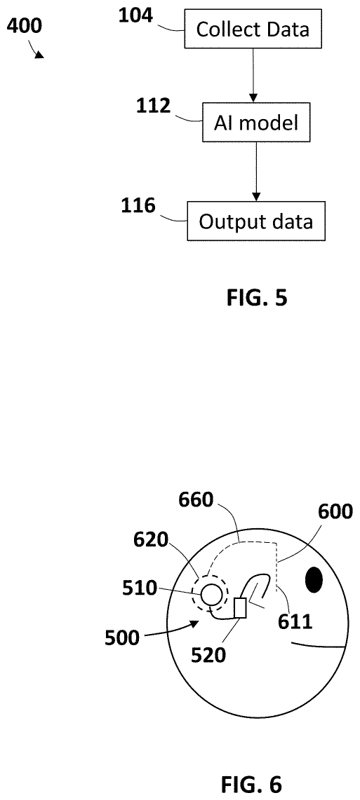

As shown in FIG. 5, a method 400 may comprise collecting data in block 104, such as EEG data, utilizing the established AI model 112, which may be an AI model that is continuing to be refined, to output data in block 116 regarding a current or future brain state based on the collected data. The output data may be used for any suitable purpose. For example, the data may be output to warn a subject that an adverse event may occur in the near future based on a predicted brain state. For example, if the subject is suffering from epilepsy, the subject may receive a warning that a seizure may soon occur. The subject may then have sufficient time to pull a car over if the subject is driving or sit or lay down if the subject is upright. The data may be output to inform a healthcare provider or caretaker that an adverse event is occurring or has occurred. In some embodiments, the data may be output to control administration of therapy, such as to modify a flow rate of a therapeutic fluid being introduced into the subject's brain, such as a CSF-containing space, based on the current or predicted future brain state.

The AI model may be developed in any suitable system. The AI model may be installed in any suitable system. In some embodiments, the AI model is developed in a system remote from a subject. In some embodiments, the AI model is installed in a system that is associated with the subject, such as in an implantable device, an ambulatory device, or a wearable device.

Referring now to FIG. 6, an implantable system configured to record and transmit iEEG signals from the brain of a subject, as well as an external apparatus 500 configured to receive the signals transmitted from the implanted system is shown. Implanted components are shown in dashed lines. The implanted system comprises a device 600, such as a catheter or lead, implanted in a subject's brain. The device 600 comprises electrodes (not shown) in proximity to a distal end 600 of the device 620. The device 600 includes, or is coupled to, a lead 660 that carries signals from the electrodes to signal apparatus 620. The signal apparatus 620 may transmit data regarding the electrical signals to the external apparatus 500. The external apparatus 500 comprises an inductive coupling component 510 that may be positioned over the signal apparatus and comprises a processing component 520 operably coupled to the inductive coupling component 510. The processing component 520 may include, among other things, a rechargeable battery and a processor. The external apparatus 500 may transmit data received from signal apparatus 620, or a processed version thereof, to suitable secondary device, such as a smartphone, personal computer, tablet, modem, or the like through any suitable platform, such as low power Bluetooth. The secondary device or external apparatus 500 may transmit data to the internet, where the data may be stored or retrieved by other computing devices as appropriate. Such computing devices may establish or refine the AI model based on the signals received by the electrodes. Alternatively or in addition, one or both of the signal apparatus 620 and external apparatus 500 may, at least in part, establish or refine the AI model based on the signals received by the electrodes.

The established AI model may then be installed on any suitable device that is in communication with the signal apparatus 620 or external apparatus 500 so that the device on which the AI model is installed may apply the AI model to signals as they are received by the electrodes of the implanted system. The device on which the AI model is installed may provide output (e.g., output data 116 as shown in FIG. 4) regarding a current or predicted future brain state based on data regarding the signals recorded by the electrodes.

Once an AI model has been established using iEEG data, the AI model may be refined or applied using brain activity data obtain from, for example, scalp EEG or MEG devices. The AI model may be applied to other subjects. That is, subjects that do not have, or did not have, recording electrodes implanted in their brain.

In some embodiments, output data (e.g., output data 116 as shown in FIG. 5) may be employed to administer therapeutic treatment to a subject suffering from a brain disease. In particular, the treatment may be administered in response to a current or predicted brain state determined by the AI model. In some embodiments, treatment may be administered using a device, drug, or combination thereof. In one example, treatment may be administered by providing an alert through a device to the subject or to a physician. In another example, treatment may be administered by infusing a drug, removing fluid, or providing electrical deep brain stimulation to the subject. In some preferred embodiments, the treatment includes administering a drug to a CSF-containing space of a brain of a subject, such as administering a drug to a cerebral ventricle, such as a lateral ventricle.

Specific treatments may be selected based on the refractory disease being treated. In one example, an anti-seizure treatment may be provided in response to detecting a seizure brain state in a subject with epilepsy.

The treatment may be provided in a continual (or periodic), reactionary, or preventative manner. Treatment may be administered continually to prevent onset of a potential brain state in a continual manner. For example, treatment may be delivered according to a schedule. Classification and forecasting analytics may not be needed for continual treatment but monitoring for toxicity may be increased.

Treatment may alternatively be administered upon onset of a brain state in a reactionary manner. Event classification analytics may be used to provide reactionary treatment. Forecasting may not be needed. Certain undesirable brain states may be shorted but may not be prevented when using reactionary treatment.

Further, treatment may be administered to the subject only, or selectively, before onset of a predicted brain state of the subject in a preventative manner. Forecasting may be used to provide predictive treatment. The need for toxicity monitoring may be decreased. Certain undesirable brain states may be prevented or avoided by using predictive treatment.

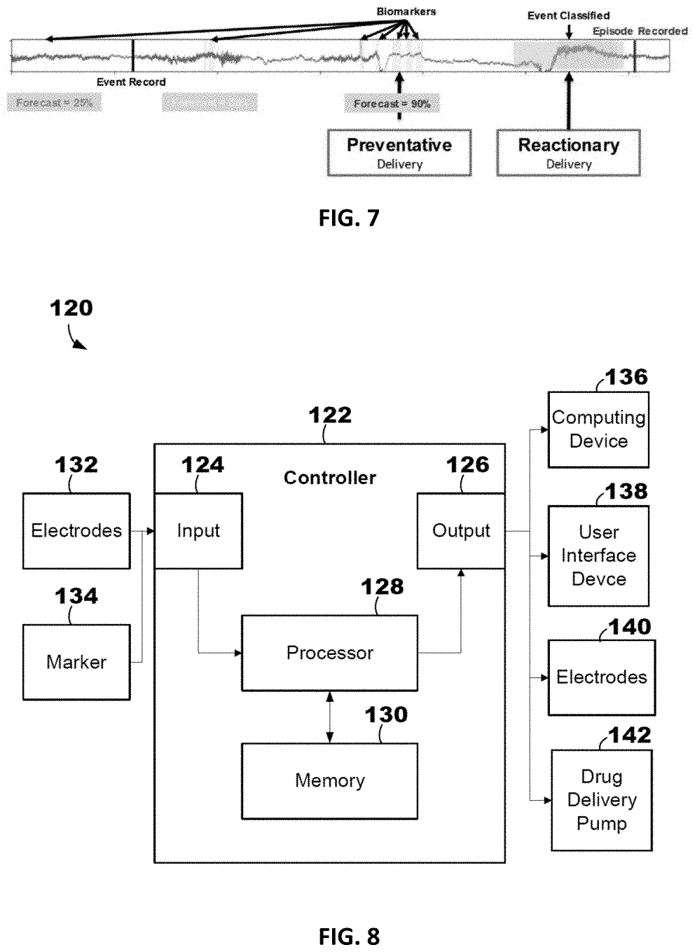

One example of the relationship between biomarkers, brain states, and different types of treatment are shown in FIG. 7. Some biomarkers may be associated with a higher probability of forecasting a brain state than other biomarkers (e.g., 90% vs. 50% vs. 25% forecast) according to the AI model.

Various types of conditions and brain states may be treated using the methods described herein. One example of conditions and brain states include post-traumatic stress disorder (PTSD). The standard of care (SOC) for severe PTSD includes psychotherapy and minimal medications. A new medication and direct intervention may be added to SOC for improvement in certain symptoms captured in the Clinician-Administered PTSD Scale (CAPS) symptom scale, primarily B symptoms. ciEEG data may be used to demonstrate improvement in such specific symptoms.

In one example, a memory may come to a subject and the subject may have a sadness or anxiety or dysphoria that occurs. The subject may press a button that is wirelessly connected to the subject's smartphone (e.g., using BLUETOOTH™). The button press may indicate a brain state, or event, that is timestamped relative to the collected ciEEG data. The experience the subject is having may reflected in broader EEG activity and may reflect mood and neuronal circuitry, which may be being monitored using the implanted one or more electrodes to provide the ciEEG data. After many events with time before and after monitored, for example, 40 of these events, a pattern may emerge to pull the event out and classify the event using the AI model. The identification of the pattern may also facilitate analysis of pre- and post-event neuronal circuitry. By filtering, and manipulating the data, features may be extracted by the AI model that not only detect the event but also allow increasingly accurate prediction of the event through the analytic and mathematical model. Once the AI model is trained, ciEEG features tied to CAPS symptoms may be used to provide an early indication of therapy treatment effect, provide information on amount, need and timing of dose titration, and facilitate “Go”/“No Go” therapy decisions for intracerebroventricular (ICV) drug treatment and personalization of oral medication and behavioral therapy including the SOC. In general, refractory PTSD subjects who suffer from repetitive distress caused by traumatic memory recall, flashbacks, and sleep disorders may be helped using the AI model. This approach and pattern of addressing spontaneous distressing events may also be applied to panic disorder which has a similar time course.

Another example of conditions, disorders, or diseases, that may be approached using the AI model is bipolar disorder, which has a different time course and pattern than PTSD. The symptom of mania is a longer episode than a memory flashback and the onset of the event more typically increases slowly over time. Similar to a memory flashback in PTSD, subjects who are having their feelings of elevated mood, increasing confidence, rush of thoughts, increasing wanting to take risks (aspects of mania), may push a button to mark or label data. EEG and feature extraction will proceed using the AI model in a similar way and will preferably reach a threshold number of events. Once trained, the AI model may be used to treat bipolar disorder, as well as depression and generalized anxiety disorders, or states, in a similar manner.

Yet another example of conditions, disorders, or diseases, that may be approached using the AI model is suicidal feelings and thoughts. While the time course and nature of thoughts are different and outcomes are different, a similar approach can be followed as described herein with respect to PTSD and bipolar disorder. Subjects may have spontaneous thoughts of suicide and spontaneous thoughts of compulsion to act on suicide or to not have insight of the temporariness of these obsessive thoughts and act upon them. When subjects have thoughts spontaneously, the thoughts may be characterized with stamping and signaling (e.g., marking and labeling). Over time, the EEG recognition will be discerned though AI model. Timely medical or social or other intervention may be enabled using this technique, which may help abort a suicide attempt or a completed suicide. Such an approach may also be used to treat addiction disorders, including for alcohol and cocaine amongst others, in a similar manner.

In some cases, a brain implant is not placed for subjects without major psychiatric disorders. In other cases, when a brain implant is used normal psychological events will occur for the subject.

Normal psychological events, such as flow (e.g., as described by Mihaily Csikszentmihalyi) may be identified. Flow is a brain state associated with optimal attention, satisfaction, and happiness that occurs when engaged in challenging and stimulating activities overlapping with attention and learning. The subject may have the ability to recognize the state and mark the events and after enough events are identified in the ciEEG, and feature extraction in the AI model may be used to identify and afterwards predict brain states or events. Such an approach may not be restricted to flow, but may include other, learning and decision-making brain states and processes.

For some subjects with brain implants, for example, when therapies are administered with a ventriculostomy catheter comprising electrodes for iEEG recording, a baseline and disrupted brain state may be determined. Feature learning in the AI model may be able to predict how medication or disease evolution, like hypertensive or hypovolemic medication for a ruptured aneurysm or hyperventilation or mannitol for elevated intracranial pressure, may be affecting brain function. Such information may allow for better titration and examination of the subject's brain function. After acute management, subjects may have issues with anxiety, agitation, and cognitive impairment. Feature learning in the AI model could be applied using collected events and population prediction after multiple subjects are monitored and AI techniques are used on ciEEG data.

In some embodiments, methods may include an iterative process in which data (e.g., EEG data) is collected (e.g., at block 104 of FIG. 1 or FIG. 5) after treatment is administered and the AI model is refined. Over time, the AI model may improve. In particular, the virtuous cycle of a trained model may continue to extract features related to events over time that describe and predict event more accurately as more data is provided to the model. Accuracy of the AI model may increase over time to more accurately diagnose monitored brain psychiatric or psychological symptoms based on the collected data.

FIG. 8 shows one example of a system 120 that may be used with a method as described herein to collect EEG data and adjust therapy delivery based on the collected EEG data through the use of an AI model. The system 120 may include a controller 122. The controller 122 may include an input interface 124, an output interface 126, a processor 128 operably coupled to the input and output, and a memory 130 operably coupled to the processor. The input interface 124 and the output interface 126 may be collectively referred to as a communication interface.

The controller 122 may be operably coupled to one or more electrodes 132 through the input interface 124. The electrodes 132 may be used to collect data, such as electrical signal data indicative of activity in white or grey matter of the brain and used as brain activity data.

One or more markers 134 may be operably coupled to the controller 122 through the input interface 124. The marker 134 may be used by the subject or physician to label data relative to the collected data.

The collected data from the input interface 124 may be stored in the memory 130. The processor 128 may use the data as input to an AI model. The AI model may be executed on the processor 128 to generate brain state data, which may also be stored in the memory.

The system 120 may include one or more devices operably coupled to the controller 122 using the output interface 126. The system 120 may include a computing device 136, a user interface device 138, an electrode 140 (e.g., to use for delivering deep brain stimulation therapy), or a drug delivery pump 142. The processor 128 may provide data or commands to devices coupled to the output interface 126 based on the brain state generated by the AI model.

One or more of the components, such as controllers, sensors or interfaces, described herein may include a processor, such as a central processing unit (CPU), computer, logic array, or other device capable of directing data coming into or out of a device, system, or component thereof. The controller may include one or more computing devices having memory, processing, and communication hardware. The controller may include circuitry used to couple various components of the controller together or with other components operably coupled to the controller. The functions of the controller may be performed by hardware and/or as computer instructions on a non-transient computer readable storage medium.

The processor of the controller may include any one or more of a microprocessor, a microcontroller, a digital signal processor (DSP), an application specific integrated circuit (ASIC), a field-programmable gate array (FPGA), and/or equivalent discrete or integrated logic circuitry. In some examples, the processor may include multiple components, such as any combination of one or more microprocessors, one or more controllers, one or more DSPs, one or more ASICs, and/or one or more FPGAs, as well as other discrete or integrated logic circuitry. The functions attributed to the controller or processor herein may be embodied as software, firmware, hardware, or any combination thereof. While described herein as a processor-based system, an alternative controller could utilize other components such as relays and timers to achieve the desired results, either alone or in combination with a microprocessor-based system.

In one or more embodiments, the functionality of systems and devices may be implemented using one or more computer programs using a computing apparatus, which may include one or more processors and/or memory. Program code and/or logic described herein may be applied to input data/information to perform functionality described herein and generate desired output data/information. The output data/information may be applied as an input to one or more other devices and/or methods as described herein or as would be applied in a known fashion. In view of the above, it will be readily apparent that the controller functionality as described herein may be implemented in any manner known to one skilled in the art.

While the present disclosure is not so limited, an appreciation of various aspects of the disclosure will be gained through a discussion of the specific examples and illustrative embodiments provided below. Various modifications of the examples and illustrative embodiments, as well as additional embodiments of the disclosure, will become apparent herein.

Example 1: A method comprising receiving electroencephalogram (EEG) data from at least one electrode implanted in a brain of a subject; and establishing an artificial intelligence (AI) model to identify an electrical signal biomarker associated with a brain state from the EEG data.

Example 2: The method of Example 1, wherein the electrical signal biomarker associated with the brain state is an electrical signal biomarker associated with a disease of the brain of the subject.

Example 3: The method of Example 1 or 2, wherein the subject has a lead or catheter implanted in the brain to treat a disease of the brain.

Example 4: The method of Example 3, wherein the subject has a catheter implanted in the brain.

Example 5: The method of Example 4, wherein the catheter comprises an external ventricular drainage catheter or a ventriculoperitoneal shunt.

Example 6: The method of Example 4 or 5, wherein the catheter comprises a therapeutic agent delivery catheter.

Example 7: The method of Example 6, wherein the therapeutic agent delivery catheter is configured to deliver a therapeutic agent to a CSF-containing space of the subject.

Example 8: The method of Example 6, wherein the therapeutic agent delivery catheter is configured to deliver a therapeutic agent to a cerebral ventricle of the subject.

Example 9: The method of any one of Examples 1 to 8, wherein the at least one electrode is implanted in white matter or grey matter of the brain.

Example 10: The method of any one of Examples 1 to 8, wherein the at least one electrode is implanted in white matter of the brain.

Example 11: The method of any one of Examples 1 to 10, comprising receiving electroencephalogram (EEG) or magnetoencephalogram (MEG) data from a device positioned on the scalp of the subject; and applying the established AI model to identify an electrical signal biomarker associated with a brain state in the electroencephalogram (EEG) or magnetoencephalogram (MEG) data.

Example 12: The method of any one of Examples 1 to 10, comprising receiving electroencephalogram (EEG) or magnetoencephalogram (MEG) data from a second subject different from the subject in which the electrode is implanted in the brain; and applying the established AI model to identify an electrical signal biomarker associated with a brain state in the electroencephalogram (EEG) or magnetoencephalogram (MEG) data of the second subject.

Example 13, The method of any one of Examples 1 to 12, comprising applying the established AI model to predict a future brain state of the subject.

Example 14, The method of Example 13, comprising notifying the subject of the predicted future brain state.

Example 15. The method of Examples 1 to 14, comprising applying the AI model to adjust administration of a therapy to the brain of the subject or the second subject based on the received data.

Example 16. The method of any one of Examples 1 to 15, wherein establishing the AI model comprises receiving labeling data to label a brain state relative to the EEG data and training a developing AI model based on the labeling data and the EEG data.

Example 17: The method of any one of Examples 1 to 16, wherein establishing the AI model comprises training a developing AI model based on unlabeled EEG data.

Example 18: The method of any one of Examples 1 to 17, wherein establishing the AI model comprises training a developing AI model using unsupervised or semi-supervised feature learning to identify electrical biomarkers associated with brain states from the EEG data.

Example 19: The method of one of Examples 1 to 18, wherein establishing the AI model comprises training a developing AI model using different data to refine the developing AI model after unsupervised or semi-supervised feature learning to train the developing AI model.

Example 20: The method of Example 19, wherein training the AI model using different data to refine the developing AI model comprises using supervised feature learning using labeled data to refine the developing AI model.

Example 21: The method of any one of Examples 1 to 20, wherein the AI model comprises a contrastive learning model.

Example 22: The method of Example 21, wherein the AI model is configured to determine one or more brain states based on a first duration between a first data segment and a second data segment of the EEG data.

Example 23: The method of Example 22, wherein the AI model is configured to determine that the first data segment and the second data segment correspond to a first brain state.

Example 24: The method of Example 23, wherein the AI model is configured to determine that a third data segment of the EEG data corresponds to a second brain state different than the first brain state in response to a second duration between the first data segment and the third data segment being greater than the first duration.

Example 25: A method comprising receiving electroencephalogram (EEG) data from at least one electrode implanted in a brain of a subject; and determining a brain state or predicting a future brain state of the subject based on the EEG data using an artificial intelligence (AI) model.

Example 26: The method of Example 25, wherein the brain state or predicted brain state is associated with a disease of the brain of the subject.

Example 27: The method of Example 25 or 26, wherein the subject has a catheter implanted in the brain to treat a disease of the brain.

Example 28: The method of Example 25, wherein the catheter comprises an external ventricular drainage catheter or a ventriculoperitoneal shunt.

Example 29: The method of Example 25 or 26, wherein the catheter comprises a therapeutic agent delivery catheter.

Example 30: The method of Example 29, wherein the therapeutic agent delivery catheter is configured to deliver a therapeutic agent to a CSF-containing space of the subject.

Example 31: The method of Example 30, wherein the therapeutic agent delivery catheter is configured to deliver a therapeutic agent to a cerebral ventricle of the subject.

Example 32: The method of any one of Examples 25 to 31, wherein the at least one electrode is implanted in white matter or grey matter of the brain.

Example 33: The method of any one of Examples 25 to 32, wherein the at least one electrode is implanted in white matter of the brain.

Example 34: The method of any one of Examples 25 to 33, wherein determining the brain state or predicting the future brain state comprising determining a brain state associated with a disease of the brain or predicting a future brain state associated with a disease of the brain.

Example 35: The method of any one of Examples 25 to 34, wherein determining the brain state or predicting the future brain state comprises determining a psychiatric brain state or predicting a future psychiatric brain state.

Example 36: The method of any one of Examples 25 to 35, comprising administering treatment to the subject in response to the current or predicted brain state in the brain state data.

Example 37: The method of Example 36, wherein treatment is administered before onset of a predicted brain state of the subject.

Example 38: The method of Example 36 or 37, wherein administering treatment comprises providing an alert to the subject, a healthcare provider, or a caretaker.

Example 39: The method of any one of Examples 36 to 38, wherein administering treatment comprises infusing a drug, removing fluid, or electrical deep brain stimulation.

Example 40: The method of any one of Examples 36 to 39, wherein the treatment comprises a treatment for one or more of the following wherein the treatment comprises a treatment for one or more of the following: epilepsy, bipolar disorder, depressive disorder spectrum, anxiety disorder spectrum including PTSD, cognitive disorder spectrum, memory disorder spectrum, processing speed disorder spectrum subarachnoid hemorrhage, intracerebral hemorrhage, subdural hemorrhage, extradural hemorrhage, obstructive hydrocephalus, nervous system cancer (such as secondary malignant neoplasm of the brain, spinal cord, or other parts of the nervous system; malignant neoplasm of the cerebellum nos; malignant lymphoma of an unspecified site, extranodal site, or solid organ site; and the like), cerebral artery occlusion, unspecified cerebral infarction, closed fracture of the base of the skull (which may be associated with one or more of subarachnoid hemorrhage, subdural hemorrhage, extradural hemorrhage, and loss of consciousness), infection and inflammatory reaction (such as due to nervous system device, implant or graft), mechanical complication of a nervous system device, implant or graft), traumatic brain injury, congenital hydrocephalus, and aneurysms.

Example 41: The method of any one of Examples 25 to 40, wherein determining the brain state or predicted future brain state comprises receiving EEG data from two or more electrodes.

Example 42: The method of any one of Examples 25 to 41, comprising receiving other data than the EEG data and wherein the other data is used in determining the brain state or predicting the future brain state.

Example 43: The method of Example 42, wherein the other data comprises one or more of the following types of data corresponding to the subject: activity, motion, heart rate, visual stimuli, audio or video recordings, and skin conductance.

Example 44: The method of any one of Examples 25 to 43, comprising receiving labeling data to label a brain state relative to the EEG data and training the AI model based on the labeling data and the EEG data.

Example 45: The method of any one of Examples 25 to 44, comprising training the AI model based on unlabeled EEG data.

Example 46: The method of any one of Examples 25 to 45, comprising training the AI model using unsupervised or semi-supervised feature learning to identify electrical biomarkers associated with brain states from the EEG data.

Example 47: The method of one of Examples 25 to 46, comprising training the AI model using different data to refine the AI model after unsupervised or semi-supervised feature learning to train the AI model.

Example 48: The method of Example 47, wherein training the AI model using different data to refine the AI model comprises using supervised feature learning using labeled data to refine the AI model.

Example 49: The method of any one of Examples 25 to 48, wherein the AI model comprises a contrastive learning model.

Example 50: The method of Example 49, wherein the AI model is configured to determine one or more brain states based on a first duration between a first data segment and a second data segment of the EEG data.

Example 51: The method of Example 50, wherein the AI model is configured to determine that the first data segment and the second data segment correspond to a first brain state.

Example 52: The method of Example 51, wherein the AI model is configured to determine that a third data segment of the EEG data corresponds to a second brain state different than the first brain state in response to a second duration between the first data segment and the third data segment being greater than the first duration.

Example 53: The method of any one of Examples 25 to 52, comprising generating a visual representation of multidimensional brain activity data in a lower-dimensional data structure; receiving labeling data to label a brain state relative to the visual representation of the lower-dimensional data structure; and training the AI model based on the labeling data.

Example 54: A device or system comprising one or more electrodes configured to be positioned on in the white or grey matter of the brain of a subject to record activity of the white or grey matter; and a controller operably coupled to the one or more electrodes configured to carry out the method according to any one of the Examples 1 to 53.

Example 55: A device or system comprising one or more electrodes configured to be positioned on the surface of the head of a subject to record electrical activity of the white or grey matter of the brain of a subject; and a controller operably coupled to the one or more electrodes configured to carry out the method according any one of Examples 1 to 53.

Example 56: The device or system of Example 54 or 55, wherein the controller is wirelessly coupled to the one or more electrodes.

Example 57: The device or system of any one of Examples 54 to 56, wherein the controller comprises at a remote computing system configured to train the AI model.

Thus, various embodiments of MONITORING BASED ON CONTINUOUS INTRACRANIAL EEG ACTIVITY are disclosed. Although reference is made herein to the accompanying set of drawings that form part of this disclosure, one of at least ordinary skill in the art will appreciate that various adaptations and modifications of the embodiments described herein are within, or do not depart from, the scope of this disclosure. For example, aspects of the embodiments described herein may be combined in a variety of ways with each other. Therefore, it is to be understood that, within the scope of the appended claims, the claimed invention may be practiced other than as explicitly described herein.

All references and publications cited herein are expressly incorporated herein by reference in their entirety for all purposes, except to the extent any aspect directly contradicts this disclosure.

All scientific and technical terms used herein have meanings commonly used in the art unless otherwise specified. The definitions provided herein are to facilitate understanding of certain terms used frequently herein and are not meant to limit the scope of the present disclosure.

Unless otherwise indicated, all numbers expressing feature sizes, amounts, and physical properties used in the specification and claims may be understood as being modified either by the term “exactly” or “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein or, for example, within typical ranges of experimental error.

The terms “coupled” or “connected” refer to elements being attached to each other either directly (in direct contact with each other) or indirectly (having one or more elements between and attaching the two elements). Either term may be replaced to “couplable” or “connectable” to describe that the elements are configured to be coupled or connected. In addition, either term may be modified by “operatively” and “operably,” which may be used interchangeably, to describe that the coupling or connection is configured to allow the components to interact to carry out functionality.

As used herein, the term “configured to” may be used interchangeably with the terms “adapted to” or “structured to” unless the content of this disclosure clearly dictates otherwise.

The term “or” is generally employed in its inclusive sense, for example, to mean “and/or” unless the context clearly dictates otherwise. The term “and/or” means one or all of the listed elements or a combination of at least two of the listed elements.

The phrases “at least one of,” “comprises at least one of,” and “one or more of” followed by a list refers to any one of the items in the list and any combination of two or more items in the list.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the disclosure.

As used herein, the term “biomarker” refers to any suitable indication of a biological process, pathogenic process, or response to exposure or intervention, including therapeutic interventions. Some biomarkers may be identified in literature, for example, as defined by the U.S. Food and Drug Administration (FDA). Different particular types of biomarkers may be used. Diagnostic biomarkers may be used to increase and optimize sensitivity and specificity for a disease in question. Predictive biomarkers may provide a new measurement tool which can provide insight into the likelihood of treatment response. Prognostic biomarkers may be compared over time to determine future disease course. Pharmacodynamic biomarkers may react in response to treatment. One or more of these different types of biomarkers may be identified in establishing an AI model or may be used by an AI model to determine one or more brain states of the subject.

What is claimed is:

1. A method comprising:
receiving electroencephalogram (EEG) data from at least one electrode implanted in a brain of a subject, wherein the subject has a catheter implanted in the brain, wherein at least a portion of the catheter is implanted in a cerebrospinal fluid (CSF)-containing space of the brain; and
determining a brain state or predicting a future brain state of the subject based on the EEG data using an artificial intelligence (AI) model.

2. The method of claim 1, wherein the brain state or predicted brain state is associated with a disease of the brain of the subject.

3. The method of claim 1, wherein the catheter comprises an external ventricular drainage catheter or a ventriculoperitoneal shunt.

4. The method of claim 1, wherein the catheter comprises a therapeutic agent delivery catheter.

5. The method of claim 1, wherein the at least one electrode is implanted in white matter of the brain.

6. The method of claim 1, wherein determining the brain state or predicting the future brain state comprising determining a brain state associated with a disease of the brain or predicting a future brain state associated with a disease of the brain.

7. The method of claim 1, wherein determining the brain state or predicting the future brain state comprises determining a psychiatric brain state or predicting a future psychiatric brain state.

8. The method of claim 1, comprising administering treatment to the subject in response to the current or predicted brain state in the brain state data.

9. The method of claim 8, wherein treatment is administered before onset of a predicted brain state of the subject.

10. The method of claim 8, wherein administering treatment comprises providing an alert to the subject, a healthcare provider, or a caretaker.

11. The method of claim 8, wherein administering treatment comprises infusing a drug through the catheter to the CSF-containing space of the brain of the subject.

12. The method of claim 8, wherein the treatment comprises a treatment for one or more of the following: epilepsy, bipolar disorder, depressive disorder spectrum, anxiety disorder spectrum including post-traumatic stress disorder (PTSD), cognitive disorder spectrum, memory disorder spectrum, processing speed disorder spectrum subarachnoid hemorrhage, intracerebral hemorrhage, subdural hemorrhage, extradural hemorrhage, obstructive hydrocephalus, nervous system cancer (such as secondary malignant neoplasm of the brain, spinal cord, or other parts of the nervous system; malignant neoplasm of the cerebellum nos; malignant lymphoma of an unspecified site, extranodal site, or solid organ site; and the like), cerebral artery occlusion, unspecified cerebral infarction, closed fracture of the base of the skull (which may be associated with one or more of subarachnoid hemorrhage, subdural hemorrhage, extradural hemorrhage, and loss of consciousness), infection and inflammatory reaction (such as due to nervous system device, implant or graft), mechanical complication of a nervous system device, implant or graft), traumatic brain injury, congenital hydrocephalus, and aneurysms.

13. The method of claim 1, wherein determining the brain state or predicted future brain state comprises receiving EEG data from two or more electrodes.

14. The method of claim 1, comprising receiving other data than the EEG data and wherein the other data is used in determining the brain state or predicting the future brain state.

15. The method of claim 14, wherein the other data comprises one or more of the following types of data corresponding to the subject: activity, motion, heart rate, visual stimuli, audio or video recordings, and skin conductance.

16. The method of claim 1, comprising receiving labeling data to label a brain state relative to the EEG data and training the AI model based on the labeling data and the EEG data.

17. The method of claim 1, comprising training the AI model based on unlabeled EEG data.

18. The method of claim 1, comprising training the AI model using unsupervised or semi-supervised feature learning to identify electrical biomarkers associated with brain states from the EEG data.

19. The method of claim 1, comprising training the AI model using different data to refine the AI model after unsupervised or semi-supervised feature learning to train the AI model.

20. The method of claim 19, wherein training the AI model using different data to refine the AI model comprises using supervised feature learning using labeled data to refine the AI model.

21. The method of claim 1, wherein the AI model comprises a contrastive learning model.
